(19) United States
(12) Patent Application Publication
(10) Pub. No.: US 2006/0211633 A1
(43) Pub. Date: Sep. 21, 2006

(54) D-mannose contraceptives

(76) Inventors: Dale L. Benedict, Fayetteville, AR (US); Martha J. Benedict, Fayetteville, AR (US)

Correspondence Address: Steven Thrasher, 391 Sandhill Dr., Richardson, TX, 75080, US

(21) Appl. No.: 11/439,403

(22) Filed: May 23, 2006

Related U.S. Application Data
(63) Continuation of application No. 10/921,748, filed on Aug. 18, 2004.
Application No. 10/921,748 is a continuation of application No. 10/231,399, filed on Aug. 29, 2002, now Pat. No. 6,753,319.

(51) Int. Cl. A61K 31/70

(52) U.S. Cl. 514/23

(57) ABSTRACT

The present invention concerns the use of D-mannose to prevent or inhibit uniting of sperm and egg/zygote in the conception process, both for specific target animals as well as general animal populations. It is emphasized that this abstract is provided to comply with the rules requiring an abstract that will allow a searcher or other reader to quickly ascertain the subject matter of the technical disclosure. It is submitted with the understanding that it will not be used to interpret or limit the scope or meaning of the claims. 37 CFR 1.72(b).

### CROSS-REFERENCE TO RELATED APPLICATIONS

This application is a continuation of and claims priority from Co-pending U.S. patent application Ser. No. 10/921,748, filed Mar. 26, 2004, entitled D-MANNOSE CONTRACEPTIVES by Dale Benedict, which is a continuation in part of now U.S. Pat. No. 6,753,319 entitled D-MANNOSE CONTRACEPTIVES, also by Dale Benedict, which was filed on Aug. 29, 2002.

### FIELD OF THE INVENTION

The invention relates to the use of D-Mannose alone and in combination with other compounds, as a specific contraceptive, and to control animal populations.

### PROBLEM STATEMENT

Interpretation Considerations

This section describes the technical field in more detail, and discusses problems encountered in the technical field. This section does not describe prior art as defined for purposes of anticipation or obviousness under 35 U.S.C. section 102 or 35 U.S.C. section 103. Thus, nothing stated in the Problem Statement is to be construed as prior art.

Discussion

The search for effective contraceptives that are both inexpensive and without undesired side effects has long been desired. The present invention concerns the use of D-Mannose administration to either male or female animals as a method of inhibiting conception specifically for a targeted animal, or generally across an animal population.

### BRIEF DESCRIPTION OF THE DRAWINGS

Various aspects of the invention, as well as an embodiment, are better understood by reference to the following detailed description. To better understand the invention, the detailed description should be read in conjunction with the drawings in which:

FIG. 1 is a chart providing indications of desired dosages of D-Mannose for various contraceptive applications.

### EXEMPLARY EMBODIMENT OF A BEST MODE

Interpretation Considerations

When reading this section (An Exemplary Embodiment of a Best Mode, which describes an exemplary embodiment of the best mode of the invention, hereinafter “exemplary embodiment”), one should keep in mind several points. First, the following exemplary embodiment is what the inventor believes to be the best mode for practicing the invention at the time this patent was filed. Thus, since one of ordinary skill in the art may recognize from the following exemplary embodiment that substantially equivalent structures or substantially equivalent acts may be used to achieve the same results in exactly the same way, or to achieve the same results in a not dissimilar way, the following exemplary embodiment should not be interpreted as limiting the invention to one embodiment.

Likewise, individual aspects (sometimes called species) of the invention are provided as examples, and, accordingly, one of ordinary skill in the art may recognize from a following exemplary structure (or a following exemplary act) that a substantially equivalent structure or substantially equivalent act may be used to either achieve the same results in substantially the same way, or to achieve the same results in a not dissimilar way.

Accordingly, the discussion of a species (or a specific item) invokes the genus (the class of items) to which that species belongs as well as related species in that genus. Likewise, the recitation of a genus invokes the species known in the art. Furthermore, it is recognized that as technology develops, a number of additional alternatives to achieve an aspect of the invention may arise. Such advances are hereby incorporated within their respective genus, and should be recognized as being functionally equivalent or structurally equivalent to the aspect shown or described.

Second, the only essential aspects of the invention are identified by the claims. Thus, aspects of the invention, including elements, acts, functions, and relationships (shown or described) should not be interpreted as being essential unless they are explicitly described and identified as being essential. Third, a function or an act should be interpreted as incorporating all modes of doing that function or act, unless otherwise explicitly stated (for example, one recognizes that “tacking” may be done by nailing, stapling, gluing, hot gunning, riveting, etc., and so a use of the word tacking invokes stapling, gluing, etc., and all other modes of that word and similar words, such as “attaching”).

Fourth, unless explicitly stated otherwise, conjunctive words (such as “or”, “and”, “including”, or “comprising” for example) should be interpreted in the inclusive, not the exclusive, sense. Fifth, the words “means” and “step” are provided to facilitate the reader's understanding of the invention and do not mean “means” or “step” as defined in §112, paragraph 6 of 35 U.S.C., unless used as “means for -functioning-” or “step for -functioning-” in the Claims section. Sixth, the invention is also described in view of the Festo decisions, and, in that regard, the claims and the invention incorporate equivalents known, unknown, foreseeable, and unforeseeable. Seventh, the foregoing discussions and definitions are provided for clarification purposes and are not limiting. Words and phrases are to be given their ordinary plain meaning unless indicated otherwise.

Description

As described in the above-referenced US Patents and US Patent Applications by Dale Benedict, D-Mannose inhibits the adherence of bacteria to membranes or cell walls.

Mr. Benedict has also discovered that a certain amount of silica prevents D-Mannose from compacting upon exposure to moisture. Thus, silica is useful to mix with D-Mannose powder prior to encapsulation.

In the equine field, Mr. Benedict discovered that D-Mannose in sufficient quantities at the appropriate times reduces the likelihood of conception by inhibiting the sperm from adhering or attaching to the egg via acrosome of the sperm. Accordingly, the invention finds application in all fields of mammal animal husbandry to prevent unwanted cross-breeding, and to also prevent unwanted unproductive periods that are caused by undesired breeding.

As discussed by Mr. Benedict in the above-referenced patent and patent application, one preferable dosage for an average adult female human would involve the ingestion of about three (3) capsules, each containing about 420 milligrams of D-Mannose about every two (2) hours beginning about a day before intercourse. This may be reduced to about 2-3 of such capsules about one hour before intercourse and about the same dosage about one hour after. An oral spray of a D-Mannose solution may also be used. The useful dose for an average adult female human would range from about 1 gram to about 10 grams of D-Mannose per day. For use as a male contraceptive, about twice the recommended dosage for a female should be given, namely about 2 to 20 grams of D-Mannose per day. This accounts for the average mass differences, absorption rates, and other metabolic factors that vary between men and women.

In one embodiment, oral administration may be used to administer D-mannose. In one embodiment, oral administration is achieved in capsule form, of D-Mannose to a female or male prior to intercourse. Such dosages and timing of their administration may vary to produce a most effective dosage regimen. Female dosages may vary with the ovulation cycle. Similarly, male dosages and administration may vary depending on sperm count, and whether or not the D-mannose is being supplemented with a second contraceptive.

While the exact mechanism of this contraceptive effect is incompletely defined, it is not essential to the understanding of the invention. Generally, it is sufficient to understand that many sperm cells adhere to the female zygote and apply an enzyme that deteriorates the coating of the egg. Eventually, the coating is sufficiently weakened to allow (generally) a single sperm penetration and DNA deposit. It is presently believed that D-mannose prevents, retards, and reduces the adhesion of sperm cells to the zygote. D-mannose may also have some as yet not understood effects on the sperm enzymes as well.

Accordingly, in one embodiment, the invention inhibits sperm-egg interaction to prevent or reduce the likelihood of conception by the presence of a sufficient quality of D-Mannose. It is believed that D-Mannose binds to sites on the sperm which are used by the sperm in its interaction with the egg in a way in which common table sugar, sucrose, and most other commonly used sugars do not. Then, the D-Mannose binds to sperm in a manner, which, at some concentration, makes the sperm less efficient in penetrating the zygote.

There is a synergistic interaction with other contraceptive applications, including contraceptives that also prevent conception by preventing sperm from penetrating the egg as well as other pathways. Thereby, otherwise independently ineffective contraceptives may, through the incorporation of D-Mannose, may result in effective contraceptive compounds. In addition, D-Mannose may be combined with common male and female aphrodisiacs (such as Viagra™) as a “honeymoon pill.”

Douches containing D-Mannose may be used alone or in combination with oral D-Mannose to inhibit effective sperm egg interaction. D-Mannose may be supplied vaginally with a diaphragm, or a condom that contains D-mannose. It may be supplied taken with separate hormonal pills or in pills, which are combined with hormonal pills. D-Mannose douches or intervaginal creams are effective means of delivering the D-Mannose particularly if used within one day before or soon after intercourse. The closer in time to the intercourse the vaginal applications are made, the more effective the application. Accordingly, soft drinks or alcohol-containing beverages supplemented with D-Mannose may be useful as a primary source of D-mannose, or as a “quick boost” to lessen the possibility of conception. Women of childbearing age not desiring to become pregnant should take a constant maintenance dose of D-Mannose.

While capsules are preferred at the present time, it is conceivable that D-Mannose may be utilized simply as a powder addition to drinks or foods. The dosages could be supplemented or replaced by a mannose-containing contraceptive jelly, douche or intravaginal cream, for example. Ultimately, the female or male should gain an internal concentration of mannose efficient to inhibit the fertilizing interaction of sperm and egg.

Animal populations often reach higher than desired and even infestation levels. News stories of residential invasions of literally millions of mice, thousands of koala bears, deer, coyotes, and other wild animals are often accompanied by lines describing the destruction of property or even the death of innocent children. These infestations are frequently treated by attacking the problem—too many animals—only after the condition of overpopulation exist. Treatment may take the form of hunting, or the setting of poisons, for example. However, setting poisons that kill these animals in mass sets additional problems of rotting corpses and exposes persons to such dangers. The invention provides the ability to selectively control animal populations before there is an overpopulation condition.

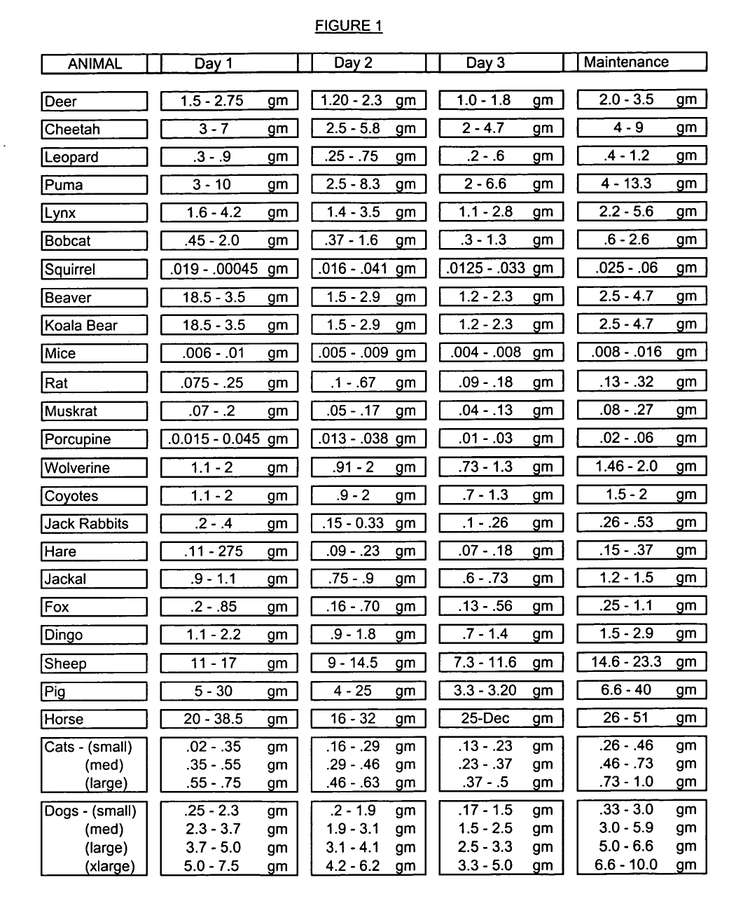

It is known that fertility levels vary from animal to animal within a species due to genetic and environmental factors. In addition, it is at times undesirable to administer a contraceptives at levels that sterilize all animals as this could have adverse consequences on the animal population that lead to species endangerment. However, dosages of D-mannose may be safely varied to from levels that absolutely prevent conception to levels that only marginally prevent conception. Accordingly, animal management may reduce a local animal population's fertility rate at a controlled rate by varying the dosage within a desired range. This is described later, below, in reference to FIG. 1.

The administration of D-Mannose to domestic or wild animals may be done to inhibit animal population growth in their natural habitats or in captivity. A specific animal that the D-Mannose is formulated for is called a targeted animal, and an animal population that a D-Mannose formulation is for is called a targeted population. Such administration to targeted animals may be in water, salt, or foods.

For example, the population of rats, mice or other rodents may be controlled by adding D-mannose to natural food forms such as seed mixtures via powder, pellet or by coating seeds with D-mannose. In addition, the population of other mammals such as coyotes, koala bears, and the like, may be controlled by adding D-mannose in powder form or as a coating spayed on or otherwise applied to berries, fruits, or other animal food. Of course, these and other animal populations can be controlled via D-mannose pellets, or liquid additive (including water, milk, or other liquid carrier), salt-licks, lick-blocks and other methods of mannose delivery systems known, unknown, foreseeable and unforseeable.

By way of further example, it is noted that many persons have observed behavior changes in their pets following having the pet spayed or neutered. Accordingly, a pet owner can be socially responsible and humane to their pet by provide D-Mannose to their pet via any of the identified delivery systems. One preferred delivery system for pets include drops that can be added to a water bowl or drinking dish, powder sprinkled over food, or directly in the food supply.

FIG. 1 illustrates a chart showing a dosage regimen for D-MANNOSE—Contraceptives to Control Animal Population. Dosages are expressed in grams, and are generally shown in recommended ranges because dosages may vary within a population based on specific animal factors, such as size, physical activity, specific absorption, and other medically significant factors. When specific animal factors are unknown (they can be learned via blood tests, or experience, for example), it is preferred to provide each animal the dosage illustrated in FIG. 1 as a high end of the recommended dosage range for each animal.

Dosages can vary in an animal population over time so that animals can be taken off D-Mannose during non-breeding seasons, and then placed back on D-Mannose either before the breeding season begins, or after some population reproduction has taken place (for example, to replenish the existing population). However, D-Mannose has no known adverse side-effects in larger dosages, and, accordingly, higher dosages are available, and may be given to animals, though there is likely to be little or no increase in efficacy.

Though the invention has been described with respect to a specific preferred embodiment, many variations and modifications will become apparent to those skilled in the art upon reading the present application. Accordingly, the providing of a higher dosage in practice necessarily incorporates a lower claimed dosage, unless otherwise indicated. It is therefore the intention that the appended claims and their equivalents be interpreted as broadly as possible in view of the prior art to include all such variations and modifications, known, unknown, foreseeable and unforeseeable.

What is claimed:

1. A method for inhibiting conception comprising:
administering D-mannose to a subject in an amount effective to inhibit interaction between a sperm and an egg; and
the D-mannose decreasing the binding between the sperm and the egg.

2. The method of claim 1 wherein the D-mannose is in an amount between about 1 gram and about 10 grams per day.

3. The method of claim 1 where D-mannose is in the form of a capsule or tablet.

4. The method of claim 1 wherein the D-mannose is in the form of a loose power and is mixed with a food or drink.

5. The method of claim 1 wherein said subject is female.

6. The method of claim 5 further comprising a supplementary D-mannose douche within one day prior to or one day after sexual intercourse.

7. The method of claim 1 further comprising use of at least one contraceptive from the group consisting of a condom, diaphragm, hormonal pill, contraceptive jelly, intravaginal cream, and douche, the D-mannose and the contraceptive being more efficient at preventing conception than either the D-mannose or the contraceptive used separately.

8. The method of claim 1 where the subject is a wild or domestic animal.

9. The method of claim 1 where D-Mannose in an amount effective to inhibit interaction between a sperm and an egg, when administered to a subject as a contraceptive.

10. A method of inhibiting conception comprising administering D-mannose to a female in an amount effective to inhibit interaction between a sperm and an egg of said female wherein the step of administering said D-mannose comprises vaginally delivering said D-mannose to said female.

11. The method of claim 10 further comprising use of at least one contraceptive selected from the group consisting of a condom, diaphragm, hormonal pill, contraceptive jelly, intravaginal cream, and douche, the D-mannose and the contraceptive being more efficient at preventing conception than either the D-mannose or the contraceptive used alone.

12. A method for inhibiting conception comprising administering D-mannose to a subject in an amount effective to inhibit interaction between a sperm and an egg of said subject wherein said D-mannose is administered in the form of a capsule or tablet, said D-mannose is administered within one day prior to or one day after sexual intercourse and wherein the administration is by means of the subject ingesting about three capsules, each capsule containing about 420 milligrams of D-mannose about every two hours beginning about a day before said intercourse, ingestion being reduced to two or three of such capsules about one hour before intercourse and about the same dosage about one hour thereafter.

13. The method of claim 12, wherein said subject is female.

14. The method of claim 12 wherein said subject is human.

15. The method of claim 12 wherein said female is a wild or domestic animal.
